(19) United States
(12) Patent Application Publication
(10) Pub. No.: US 2026/0002105 A1
(43) Pub. Date: Jan. 1, 2026

(54) LAMINAR FLOW INCORPORATED SCAFFOLD-BASED 3D CELL CULTURES

(71) Applicant: The University of North Carolina at Chapel Hill, Chapel Hill, NC (US)

(72) Inventors: Matthew Ryen Lockett, Chapel Hill, NC (US); Zachary Ryan Sitte, Chapel Hill, NC (US); Tyler Larson, Carrboro, NC (US); Thomas DiProspero, Pittsford, NY (US)

(73) Assignee: The University of North Carolina at Chapel Hill, Chapel Hill, NC (US)

(21) Appl. No.: 19/132,005

(22) PCT Filed: Nov. 22, 2023
(86) PCT No.: PCT/US2023/080958

§ 371 Date: May 22, 2025

Related U.S. Application Data
(60) Provisional application No. 63/427,266, filed on Nov. 22, 2022.

(51) Int. Cl. C12M 1/32; C12M 1/00; C12M 1/12

(52) U.S. Cl. CPC C12M 23/12; C12M 25/06; C12M 25/14; C12M 29/10

(57) ABSTRACT

A cell culture system includes at least one well plate configured to receive a cell culture wherein the at least one well plate comprises a plurality of apertures configured to provide a laminar flow of media. The cell culture system further includes a face plate, wherein the face plate is configured to cover the at least one well plate. A cell culture region is between the at least one well plate and the face plate, wherein the at least one well plate and the face plate are sealably connected to form a sealed chamber for the cell culture region where the cell culture is contained and is proximal to the laminar flow of media. The system provides a continuous laminar flow of media to the cell culture.

### CROSS REFERENCE TO RELATED APPLICATIONS

This application claims the benefit of and priority to U.S. Provisional Patent Application Ser. No. 63/427,266 entitled “LAMINAR FLOW INCORPORATED SCAFFOLD-BASED 3D CELL CULTURES,” filed Nov. 22, 2022, the disclosure of which is incorporated herein by reference in its entirety.

### GOVERNMENT INTEREST

This invention was made with government support under Grant No. GM128697 awarded by the National Institutes of Health. The government has certain rights in the invention.

### TECHNICAL FIELD

The presently disclosed subject matter generally relates to cell culture models. In particular, certain embodiments of the presently disclosed subject matter relate to continuous fluid delivery systems simulating laminar flow and methods for producing the same.

### BACKGROUND

Three-dimensional (3D) culture models incorporate tissue-like architectures and microenvironments that are more physiologically relevant than maintaining cells as monolayers on plasticware. Despite many examples of culturing cells in a 3D environment, most models rely on static culture conditions in which diffusion-dominated environments occur. These environments result in pericellular hypoxia as the rate of oxygen consumption outpaces its rate of diffusion.

### SUMMARY

This summary lists several embodiments of the presently disclosed subject matter, and in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In an aspect of the presently disclosed subject matter, there is provided a cell culture system, the system comprising at least one well plate configured to receive a cell culture wherein the at least one well plate comprises a plurality of apertures configured to provide a laminar flow of media; a face plate, wherein the face plate is configured to cover the at least one well plate; and a cell culture region between the at least one well plate and the face plate, wherein the at least one well plate and the face plate are sealably connected to form a sealed chamber for the cell culture region where the cell culture is contained and is proximal to the laminar flow of media, wherein the system provides a continuous laminar flow of media to the cell culture.

In some embodiments, the face plate is affixed to the at least one well plate.

In some embodiments, the system further comprising an O-ring, wherein the O-ring provides a seal between the face plate and the at least one well plate defining the cell culture region.

In some embodiments, the plurality of apertures comprises a plurality of tube fittings directing the laminar flow of media over the cell culture.

In some embodiments, the cell culture is positioned between the at least one well plate and/or face plate.

In some embodiments, the cell culture is embedded in a cell-compatible matrix, e.g., a hydrogel, optionally wherein the cell culture comprises a cell-laden hydrogel.

In some embodiments, the cell culture region confines the cell culture to a specific location on the at least one well plate and/or face plate.

In some embodiments, the system further comprising a porous filter separating the cell culture from the laminar flow of media.

In some embodiments, the at least one well plate and the cell culture are attached to a second at least one well plate and a second cell culture with a cell-to-cell porous membrane separating the cell culture from the second cell culture.

In some embodiments, the system comprises a first laminar flow of media connected to the at least one well plate and the first laminar flow of media connected to the second at least one well plate.

In some embodiments, the system comprises a first laminar flow of media connected to the at least one well plate and a second laminar flow of media connected to the second at least one well plate.

In some embodiments, the cell culture region comprises an exchange of the laminar flow of media after about 1 mL to about 50 mL of a media flows through the system.

In some embodiments, the cell culture region comprises an exchange of the laminar flow of media after about 20 ml of a media flows through the system.

In some embodiments, the system comprises an optimum flow rate of about 1 mL/min to about 50 mL/min for the laminar flow of media.

In some embodiments, the system comprises an optimum flow rate of about 10.185 mL/min for the laminar flow of media.

In an aspect of the disclosed subject matter, there is provided a method of continuous flow delivery to a cell culture. In some embodiments, the method comprises providing the cell culture system comprising at least one well plate configured to receive a cell culture wherein the at least one well plate comprises a plurality of apertures configured to provide a laminar flow of media; a face plate, wherein the face plate is configured to cover the at least one well plate, and a cell culture region between the at least one well plate and the face plate, wherein the at least one well plate and the face plate are sealably connected to form a sealed chamber for the cell culture region where the cell culture is contained and is proximal to the laminar flow of media, wherein the system provides a continuous laminar flow of media to the cell culture; and flowing the laminar flow media to and/or from the cell culture region.

In an aspect of the disclosed subject matter, there is provided any and all compositions, devices, systems, apparatuses, uses, and/or methods shown and/or described expressly or by implication in the information provided herewith, including but not limited to features that may be apparent and/or understood by those of skill in the art.

In some embodiments of the methods, the methods further comprise additionally flowing a first media to a first cell culture region and the first media to a second cell culture region.

In some embodiments of the methods, the methods further comprise additionally flowing a first media to a first cell culture region and a second media to a second cell culture region.

In some embodiments of the methods, the first cell culture region is separated from the second cell culture region by a cell-to-cell porous membrane.

In some embodiments of the methods, the face plate is affixed to the at least one well plate.

In some embodiments of the methods, the methods further comprise providing an O-ring, wherein the O-ring provides a seal between the face plate and the at least one well plate defining the cell culture region.

In some embodiments of the methods, the plurality of apertures comprises a plurality of tube fittings directing the laminar flow of media over the cell culture.

In some embodiments of the methods, the cell culture is embedded in a cell-compatible matrix, e.g., a hydrogel, optionally wherein the cell culture comprises a cell-laden hydrogel.

In some embodiments of the methods, the cell culture region confines the cell culture to a specific location on the at least one well plate and/or face plate.

In some embodiments of the methods, the cell culture region confines the cell culture to a specific location on the at least one well plate and/or face plate.

In some embodiments of the methods, the methods further comprise providing a porous filter separating the cell culture from the laminar flow of media.

In some embodiments of the methods, the cell culture region comprises an exchange of the laminar flow of the media after about 1 mL to about 50 mL of a media flows through the system.

In some embodiments of the methods, the cell culture region comprises an exchange of the laminar flow of the media after about 20 ml of a media flows through the system.

In some embodiments of the methods, the laminar flow of media comprises an optimum flow rate of about 1 mL/min to about 50 mL/min.

In some embodiments of the methods, the laminar flow of media comprises an optimum flow rate of about 10.185 mL/min.

In some embodiments of the methods, the face place is optically transparent.

Accordingly, it is an object of the presently disclosed subject matter to provide continuous fluid delivery systems simulating laminar flow and methods for producing the same.

These and other objects are achieved in whole or in part by the presently disclosed subject matter. Further, representative objects of the presently disclosed subject matter having been stated above, other objects and advantages of the presently disclosed subject matter will become apparent to those skilled in the art after a study of the following Description, Figures, and Examples.

### BRIEF DESCRIPTION OF THE DRAWINGS

The presently disclosed subject matter can be better understood by referring to the following Figures. The components in the Figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the Figures and the following description. The Figures are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 shows a pictorial representation of blood flowing through blood vessels under a laminar flow profile with an exemplary representation of tissue components to which the blood may pass and provide nutrients.

FIG. 2A shows a colorimetric evaluation of complete media exchange in a continuous flow delivery system (CFDS) after approximately 20 ml of medium while not creating any diffusion-limited regions indicating that the entire culture area can be utilized.

FIG. 2B shows a quantitative evaluation of the complete removal of the medium by significant removal of fluorescent solution after 20 ml of flushing water. FIG. 2C shows fluorescent solution with 5 μM calcein in the CFDS. FIG. 2D shows remnants of the fluorescent solution in an O-ring of CFDS after H₂O flushes the 5 μM calcein.

FIG. 3 shows a finite element simulation rendering to determine the flow profile in the culture device with streamlines and arrow lines representing velocity fields.

FIG. 4 shows finite element simulation used to calculate the Reynolds Number in the culture device.

FIG. 5 provides a micrograph of the MDA-MB-231 breast adenocarcinoma cell line after 72 hours in the CFDS, stained with calcein-AM (live cells) and DRAQ-5 (all cells).

FIG. 6A shows a cross sectional schematic of a CFDS with two culture scaffolds separated by a porous membrane that allows the exchange of nutrients between the scaffolds while keeping them physically separated.

FIG. 6B is a chart of relative luminous units for side 1 and side 2 of the culture scaffolds, which were removed from the assembled CFDS and assessed for cellular viability (read as luminous units).

FIGS. 7A-7C show a single CFDS while also incorporating endothelial cells and a basement membrane mimic to generate models that better recapitulate an in vivo environment.

FIGS. 8A-8B are micrograph images of endothelial cells cultured on a sheet of nylon filter paper, stained with calcein-AM (shown in blue when viewed in color) and DAPI (shown in red when viewed in color).

FIG. 8C shows a schematic demonstrating endothelial cells on the nylon filter paper exposed to flowing medium.

FIG. 9 shows a top, side and perspective views of an exemplary O-ring.

FIG. 10 shows an end, side and perspective views of an exemplary tube fitting.

FIG. 11 shows top, bottom, sides, cross-sectional and perspective views of an exemplary of at least one well plate.

FIG. 12 shows top, bottom, sides, cross-sectional and perspective views of an exemplary a cell culture region of the at least one well plate with a plurality of apertures configured to provide a laminar flow of media.

FIG. 13 shows top, side and perspective views of an exemplary face plate.

FIG. 14 is a flow diagram illustrating an example method of continuous flow delivery to a cell culture.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a,” “an,” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Unless otherwise indicated, all numbers expressing quantities of time, temperature, weight, concentration, volume, strength, speed, length, width, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±20% or ±10%, in another example ±5%, in another example ±1%, and in still another example ±0.1% from the specified amount, as such variations are appropriate to perform the disclosed methods.

Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

As used herein, the term “sealably” refers to capable of sealing or being sealed.

As used herein, the term “soluble molecule” refers to any molecule that can form a solution in an aqueous medium including but not limited to inorganic salts, organic molecules, small molecule drugs, biologics or biomolecule-based drugs, signaling molecules, chemokines, and biological molecules or synthetic mimics of biological molecules including proteins, peptides, antibodies, oligosaccharides, lipids, and/or oligonucleotides.

As used herein, the terms “transparent,” “optically transparent,” “optically clear” and “optically cleared” are used interchangeably. These terms refer to performing microscopy directly though a material because the material has a same or similar index of refraction as contents inside/on the material, e.g, cells and/or cell-compatible matrix.

As used herein, the term “cell-compatible matrix” refers to a material that supports cellular growth, proliferation, vascularization, and/or differentiation of the cells into tissue, and/or is not detrimental to cellular growth, proliferation, vascularization, and/or differentiation of the cells into tissue.

As used herein, the term “cell-compatible material” refers to a material or substrates that are sufficient to support cellular growth or proliferation and/or are not detrimental to cellular growth or proliferation.

As used herein, the terms “cell to cell interaction” and “cell to matrix interaction” can refers in some embodiments to cellular invasion through cell-compatible matrices and cell compatible porous materials, with where the cells engage other cells and/or where chemical cellular signaling that can pass through the cell-compatible matrices and cell compatible porous materials to other scaffold cups.

3D culture methods have been gaining traction as a more physiologically relevant in vitro culture method. However, many of the developed 3D culture models rely on static culture conditions or turbulent flow-driven diffusion to deliver nutrients to cells, reducing the physiological relevance of the in vitro method. Physiologically, nutrients are delivered to tissues through a combination of flow and diffusion. These flows can be laminar or turbulent, with the latter inducing convection. In addition, the nutrients often must diffuse through a layer of endothelial cells and the corresponding basement membrane before reaching a tissue, often missing in 3D culture models. Here, a more physiologically relevant nutrient delivery system for 3D cultures has been developed, with an application in some embodiments on paper-based cultures, although the disclosed systems and methods are applicable to other 3D culture systems and methods.

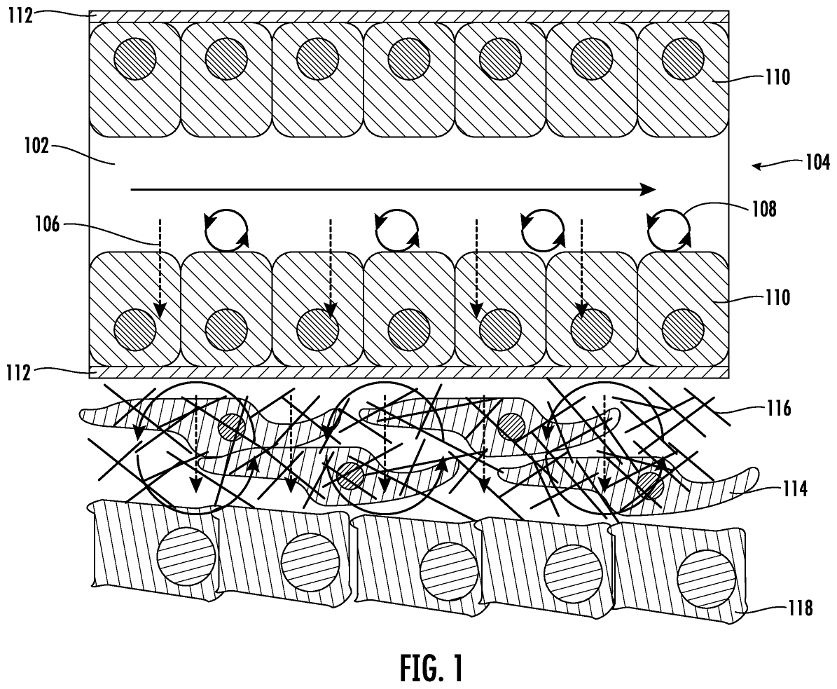

FIG. 1 shows a pictorial representation of blood 102 flowing through blood vessels 104 under a laminar flow profile with an exemplary representation of tissue components to which the blood may pass and provide nutrients via diffusion 106 and convection 108. Tissue components illustrated include endothelial cells 110, a basement membrane 112, fibroblast 114, extra cellular matrix proteins 116, and cancer cells 118. Physiologically, blood 102 flows through blood vessels 104 under turbulent and laminar flow profiles. Laminar profiles are found in the middle of the blood vessels, with turbulent flow resulting in a disrupted path or abnormality in vessel structure. Blood vessels 104 comprise a basement membrane 112 tube, with pore sizes of approximately 0.1 μm, lined with endothelial cells 110. The nutrients in blood 102 then transition out of blood vessels 104 to the surrounding cells via a combination of diffusion 106 and convection 108, induced by both the laminar flow and interstitial pressure outside of the blood vessel. The flow of nutrients through the cell-containing regions is diffusion-based, which makes the CFDS more tissue-like. Many traditional microfluidic devices with few fabrication steps, for example organ on chip devices, rely on laminar flow to do most of the exchange with a single layer of cells.

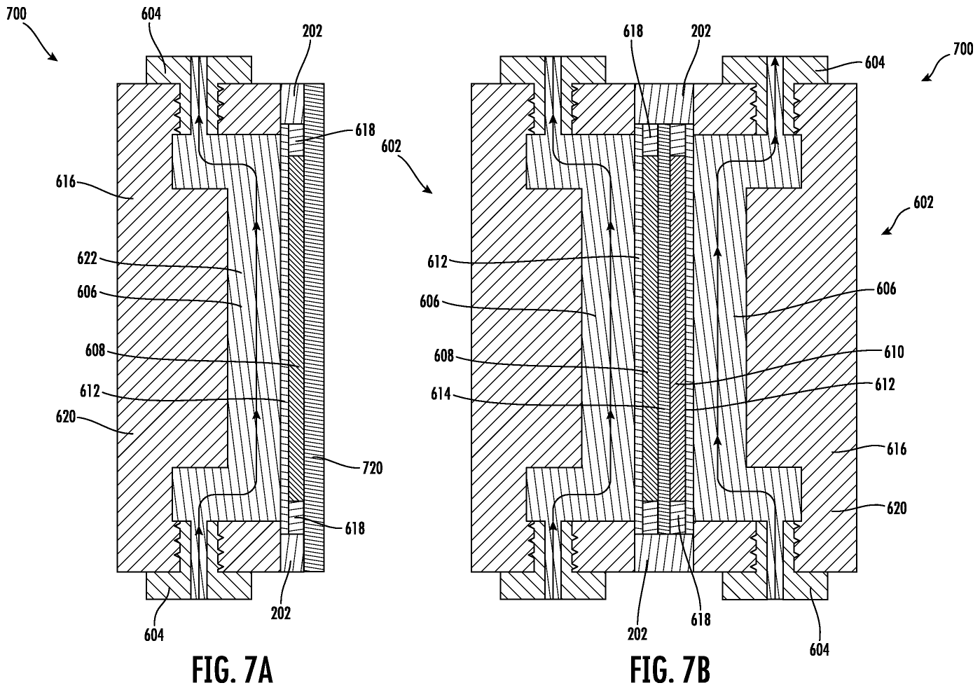

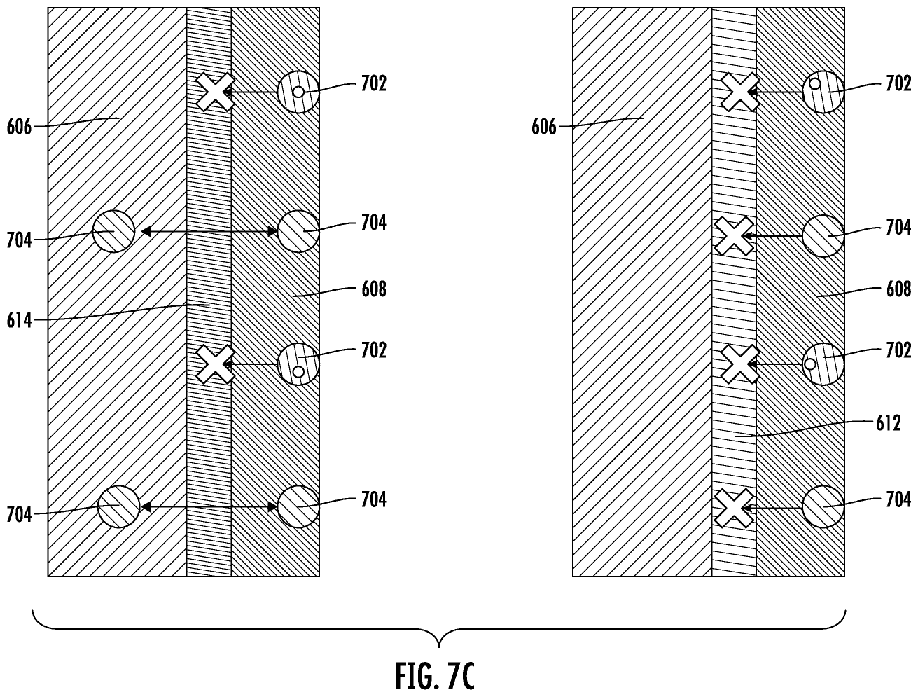

Methods, devices, and systems for providing continuous flow delivery systems (CFDSs) are disclosed. FIGS. 7A-7C show a single CFDS 700 while also incorporating endothelial cells and a basement membrane mimic to generate models that better recapitulate an in vivo environment (FIG. 7A). In an aspect of the presently disclosed subject matter, there is provided a cell culture system. A cell culture system can include any CFDS as described herein. CFDS 700 is a modular system that allows for multiple experimental set-ups previously unattainable by varying the culture holder and basement membrane mimicking material, such as dialysis sheet 612 and filter paper 614 shown in FIG. 7C, which are size-selective barriers. As shown in FIG. 7B, CFDS 700 uses linear laminar flow to deliver nutrients to 3D cell cultures (which can be provided in a cell-containing scaffold comprising a cell-compatible matrix), such as M231 cell-containing scaffold 608 and cell-containing scaffold 610. As an example, cell-containing scaffold 608 can include M231 cells and cell-containing scaffold 610 can include RMF cells. In addition to cells, cell-containing scaffolds 608 and 610 can optionally include cell-compatible matrix. CFDS 700 can also incorporate endothelial cells and a material that mimics a basement membrane to generate models that better recapitulate an in vivo environment as shown in FIG. 7A. FIG. 7C shows example configurations of the cell culture system with the system on the left including filter paper 614 as a basement membrane mimicking material to separate cell media 606 and cell-containing scaffold 608 carrying cells 702 (e.g., M231 cells) and signaling molecules 704. Filter paper 614 blocks cells 702 but allows signaling molecules 704 to pass through. The cell culture system on the right includes dialysis sheet 612 as a basement membrane mimicking material that blocks both cells 702 and signaling molecules 704. Notably, it is understood that M231 cells and RMF cells are examples of cells in the described subject matter and can be interchangeable with epithelial, cancerous, or stromal cells unless otherwise indicated without affecting the scope of the subject matter.

In some embodiments, the system comprises at least one well plate 620 configured to receive a cell culture wherein the at least one well plate 620 comprises a plurality of apertures, which may include tube fittings 604, configured to provide a laminar flow of media through a pathway 622. The cell culture system can include a face plate 720, wherein face plate 720 is configured to cover well plate 620; and a cell culture region between the at least one well plate and the face plate, wherein the at least one well plate and the face plate are sealably connected to form a sealed chamber for the cell culture region where the cell culture is contained and is proximal to the laminar flow of media, wherein the system provides a continuous laminar flow of media to the cell culture. In some embodiments, the cell culture region is defined by a border. In some embodiments the cell culture region is defined by wax borders 618. Notably, face plates described herein such as face plate 720 can be transparent, allowing for live cell imaging and microscopy.

CFDS 700 can in some embodiments utilize simple and inexpensive small-scale fabrication techniques (e.g., 3D printing and laser cutting) lowering the activation barrier of incorporating such a delivery system into already established cultures. In addition, this culture system and method allow for multiple cultures to be put in series, allowing for multiple replicates to be performed simultaneously or for multiple organ models to be placed in series to evaluate the effect of chemotherapeutics on multi-organ systems in a single experiment. Also, this modeling method represents the first generation in a series aimed at developing a small-scale home model (approximately 1000× smaller, often referred to as a milli-Human model), including liver, lung, heart, colon, and kidney models, that incorporate many physiologically relevant aspects (e.g., turnover of the blood, oxygen-carrying molecules, and central reservoir for medium) currently missing in many 3D cell culturing methods.

In some embodiments, the system further comprises a porous filter separating the cell culture from the laminar flow of media. In some embodiments, the porous filter comprises a porous membrane. In some embodiments, the porous membrane comprises a basement membrane mimicking material, for example dialysis sheet 612 or filter paper 614 as shown in FIG. 7C. In some embodiments, the porous filter comprises a material selected from the group consisting of cellulose acetate, cellulose and plant-derived materials, collagen, nitrocellulose, nylon filters (porous), nylon mesh (woven), polyamide, polyamide acrylate (PMNA), polycarbonate, polyester, polyethersulfone (PES), polylactic acid, polypropylene (porous), rayon and any combination thereof.

In some embodiments, the porous filter comprises a pore size of ranging from about 0.1 μm to about 50 μm. In some embodiments, the porous filter comprises a pore size of ranging from about 0.1 μm to about 20 μm. In some embodiments, the porous filter comprises a pore size of ranging from about 0.1 μm to about 18 μm. In some embodiments, the porous filter comprises a pore size of a range from about 7 μm to about 18 μm. In some embodiments, the pore size comprises a pore size that inhibits cellular invasion and/or migration. In some embodiments, the pore size comprises a pore size that allows cellular invasion and/or migration.

In some embodiments, the cell culture is positioned between the at least one well plate and/or face plate. In some embodiments, the cell culture is positioned on well plate 620. In some embodiments, the cell culture is positioned on face plate 620. In some embodiments, the cell culture transverses well plate 620 and face plate 720.

In some embodiments, the cell culture is embedded in a cell-compatible matrix, e.g., a hydrogel, optionally wherein the cell culture comprises a cell-laden hydrogel. In some embodiments, the cell-compatible matrix comprises a material selected from the group consisting of protein-based gels, saccharide-derived gels, glycocalyx-derived gels, synthetic hydrogels and any combination thereof. In some embodiments, the cell-compatible matrix comprises a material selected from the group consisting of collagen, fibrin, laminin, fibronectin, vitronectin, silk, silk fibroin, decellularized extracellular matrices, decellularized extracellular matrices from specific tissues, Xylyx® bio tissue-specific extracellular matrix, protein extract from Engelbreth-Holm-Swarm (EHS) culture, ATCC® basement membrane extract, Cultrex® basement membrane extract, Matrigel® basement membrane extract, MaxGel™ basement membrane extract, GrowDex® basement membrane extract, ObaGel™ basement membrane extract; alginate, agarose, chitosan, gelatin, hyaluronic acid, nanocellulose, CorGel™ gel, GrowDex® saccharide or glycocalyx-derived gel, peptide-based hydrogels, polyethylene glycol (cross-linked) gels, polyacrylamide, polyacrylate and poly-methacrylate, polyvinyl alcohol, poly(lactic-co-glycolic acid), polylactic acid, gelatin methacrylate (GelMa), poly(ethylene glycol) diacrylate (PegDa) and any combination thereof.

In some embodiments, the cell culture region confines the cell culture to a specific location on the at least one well plate 620 and/or face plate 720. In some embodiments, the cell culture region confines the cell-compatible matrix to a specific location on well plate 620 and/or face plate 720.

In some embodiments, a first at least one well plate 620 and the cell culture are attached to a second at least one well plate 620 and a second cell culture with a cell-to-cell porous membrane separating the cell culture from the second cell culture. In some embodiments, a first laminar flow of cell media 606 connected to the at least one well plate 620 and the first laminar flow of media connected to the second at least one well plate 620. In some embodiments, a first laminar flow of cell media 606 connected to the at least one well plate 620 and a second laminar flow of cell media 606 connected to the second at least one well plate 620.

In some embodiments, the cell culture region comprises an exchange of cell media 606, where the flow is laminar after about 1 mL to about 50 mL of the cell media 606 flows through the system. In some embodiments, the cell culture region comprises an exchange of cell media 606, where the flow is laminar after about 20 mL of the cell media 606 flows through the system. In some embodiments, an optimum flow rate of about 1 mL/min to about 50 mL/min for the laminar flow of media. In some embodiments, an optimum flow rate of about 10.185 mL/min for the laminar flow of cell media 606.

In some embodiments, the laminar flow of media comprises a media, such as cell media 606. In some embodiments, the media comprises any media one skilled in the art may use to support cell culture, storage and/or proliferation. In some embodiments, the media comprises universal culture media. In some embodiments, the media comprises blood mimic media. In some embodiments, the media comprises media with or without supplements. In some embodiments, the media is selected from the group consisting of glutamine, high glucose concentrations, low glucose concentrations, HEPES antibiotics, PenStrep, antimycotics, sodium pyruvate, sodium bicarbonate, Dulbecco's Modified Eagle Medium, RPMI 1640 Media, DMEM/F12 Media Formulations, Minimum Essential Medium (MEM) Ham's F-10 Media Formulation, Ham's F-12 Media Formulation, Medium 199 (M199), Eagle's Minimum Essential Medium, Iscove's Modified Dulbecco's Medium, Hybri-Care Medium, McCoy's 5A, Leibovitz's L-15 Medium and any combination thereof.

In some embodiments, well plate 620 and face plate 720 are made by a manufacture process. In some embodiments, the manufacture process is selected from the group consisting of additive manufacturing technologies, 3D printing, deposition molding, extrusion printing, photopolymerization, selective laser sintering, laser cutting, stereolithographic (SLA) printing, compression molding, injection molding, laser cutting, physical cutting, slicing, dye punching, hole punching, scissors, exact-o blading, rubber molding, thermal press with dyes, thermal press with stamps, transfer molding and any combination thereof. Representative techniques are also described in the Examples presented herein below.

In some embodiments, well plate 620 and the face plate 720 comprise a cell compatible material. In some embodiments, the cell compatible material is selected from the group consisting of nitrile (BUNA-N) rubber, ethylene propylene diene monomer (EPDM), fluorocarbon-based rubber, viton, food-grade silicone, nitrile, polydimethisiloxane (PDMS), polyethylene, high density polyethylene (HDPE), low density polyethylene (LDPE), polyetherketone (PEEK), polyethylene terephthalate (PET), polyethylene terephthalate glycol (PETG), polylactic acid, polypropylene, polystyrene, polytetrafluoroethylene (PTFE), Santoprene rubber, silicone and any combination thereof.

In some embodiments, face plate 720 is affixed to well plate 620. In some embodiments, face plate 720 is affixed to well plate by at least one connector, such as a screw or clasp.

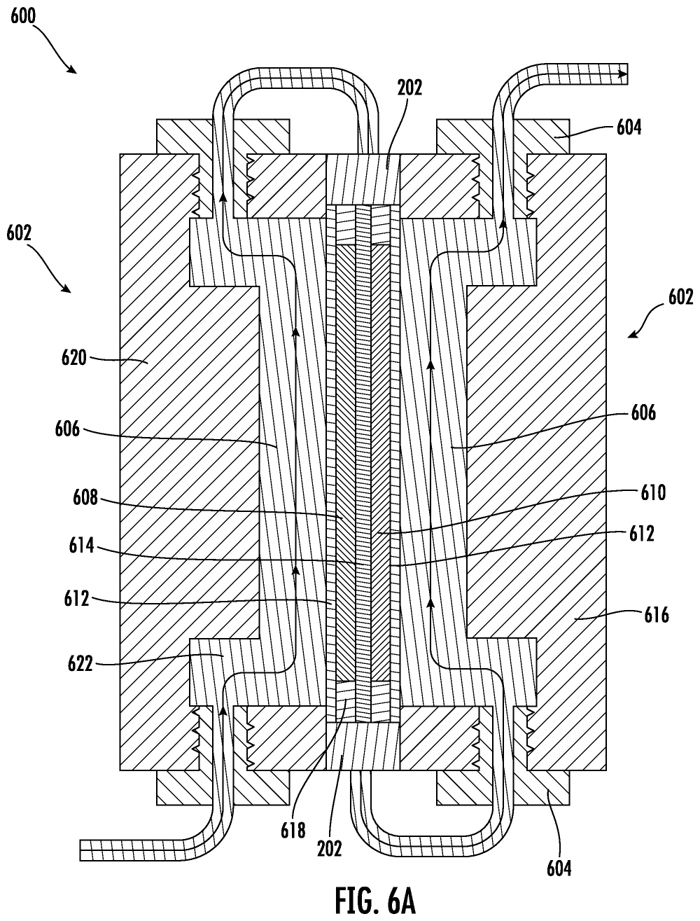

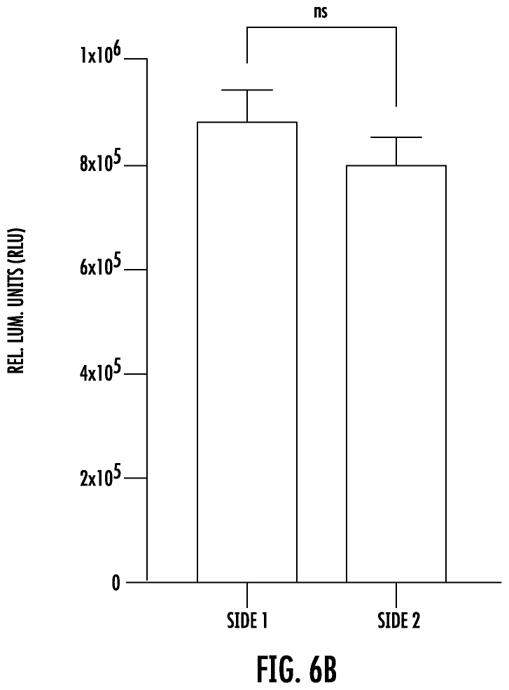

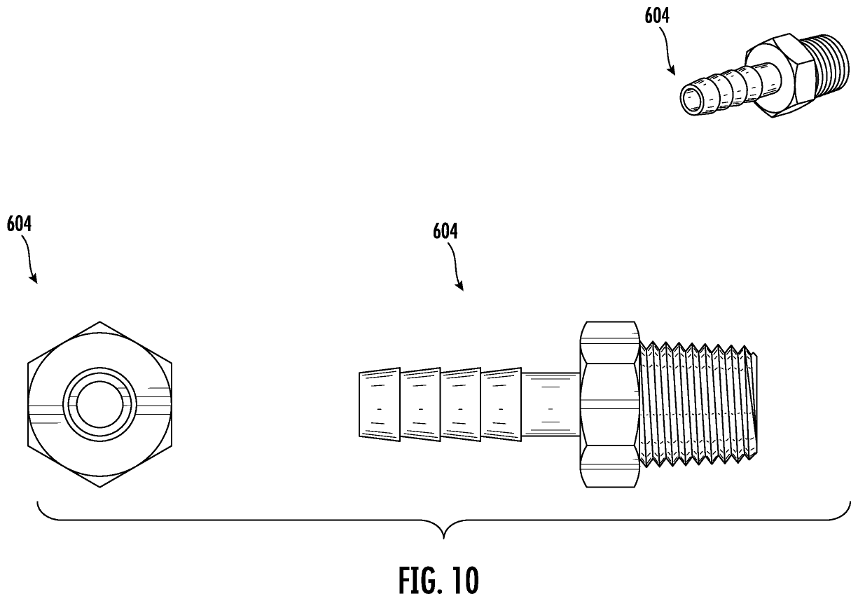

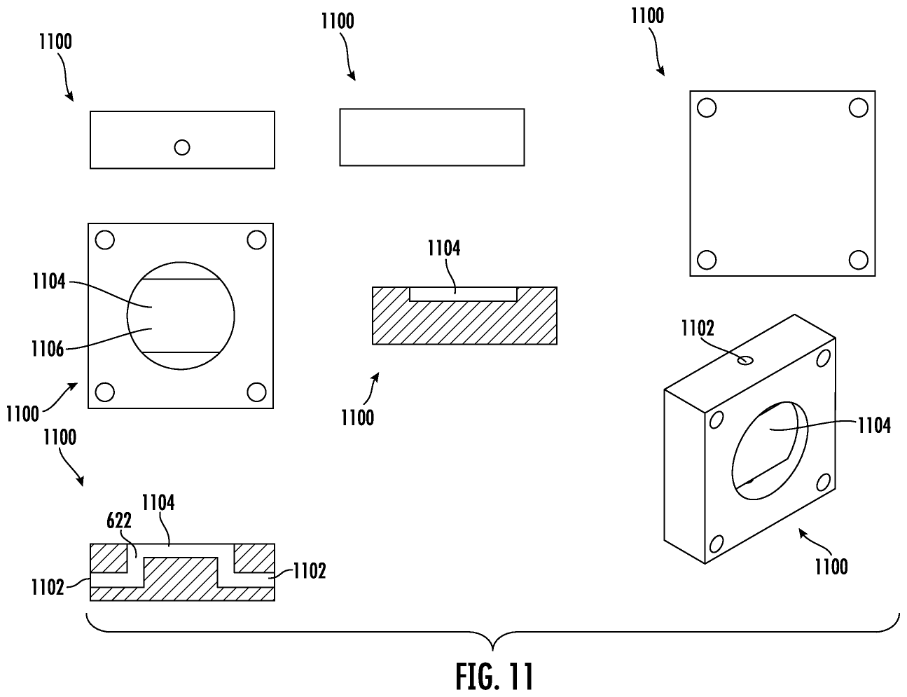

FIG. 10 shows an end, side and perspective views of an exemplary tube fitting 604. In some embodiments, the plurality of apertures 1102 comprises a plurality of tube fittings 604 (see FIGS. 6A, 7A-7C, & 10) directing the laminar flow of media over the cell culture. In some embodiments, tube fittings 604 are configured to receive a tube providing the laminar flow of media. Tube fitting 604 can have a hexagon top of about 0.600 inches from opposite edges. The hexagon top of tube fittings 604 can have a center well with a diameter of about 0.201 inches. Tube fittings 604 can have a length of about 1.678 inches. Threads can extend from a first side of the hexagon top with about 18 threads per inches, a 0.40-inch thread engagement, and ¼ NPT pipe size. A tube receiver, configured to receive a tube, can extend from an opposite side of the hexagon top with a diameter of about 0.306 inches for a ¼ inch tube ID.

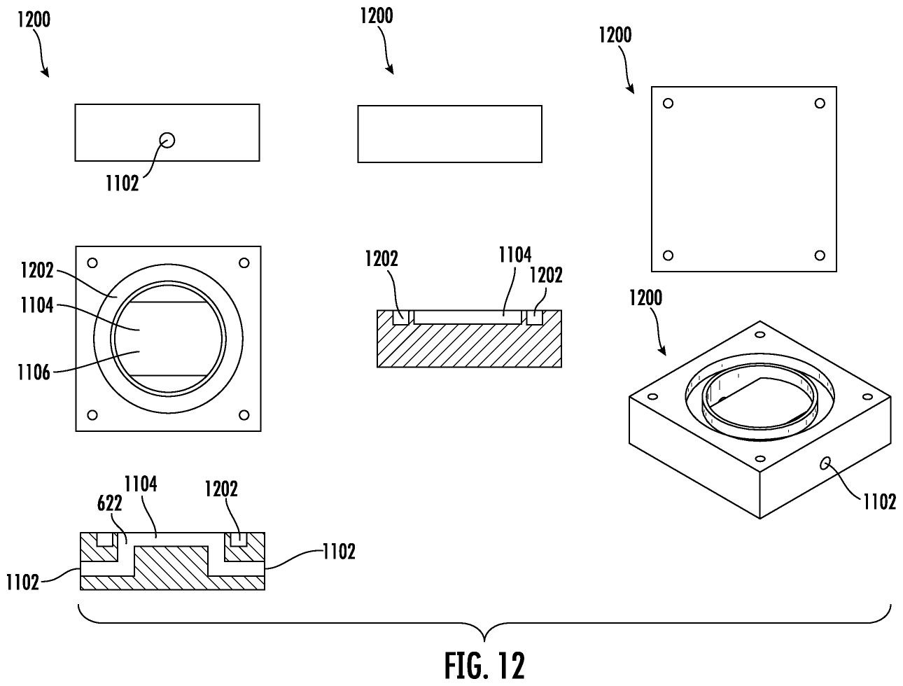

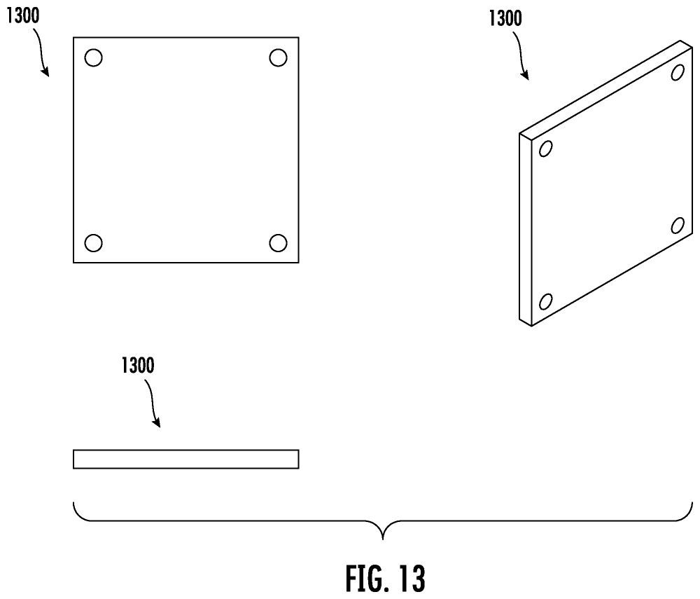

FIG. 9 shows a top, side and perspective views of an exemplary O-ring 202. In some embodiments, the system further comprises an O-ring 202, wherein the O-ring 202 provides a seal between a face plate 1300 (FIG. 13) and the at least one well plate, such as face plate 1100 shown in FIG. 11 or face plate 1200 shown in FIG. 12, defining a cell culture region 1106. O-ring 202 can have an outer diameter of about 1.25 inches, an inner diameter of about 1.0 inch, and a thickness or height of about ⅛ of an inch. In some embodiments, O-ring 202 comprises a cell-compatible material. In some embodiments, O-ring 202 comprises a cell-compatible material selected from the group consisting of BUNA-A rubber, fluorocarbon-based rubbers, Viton, food-grade silicon, PDMS, polypropylene, Santoprene rubber, silicone. In some embodiments, O-ring 202 comprises silicone.

In some embodiments, such as with well plate 1100 shown in FIG. 11, O-ring 202 is positioned on a top surface of the well plate 1100 and, having a larger diameter than well 1104, surrounds the well 1104. In some embodiments, such as with well plate 1200 shown in FIG. 12, the well plate 1200 includes a recess 1202 in the top surface to receive O-ring 202 such that the O-ring 202 is at least partially recessed in the top surface of the well plate 1200 and partially protrudes from the top surface to contact and create a seal with face plate 1300.

FIG. 11 shows top, bottom, sides, cross-sectional and perspective views of an exemplary of at least one well plate 1100. Well plate 1100 can have a square footprint with a length and width of about 40 mm and a thickness or height of about 12.38 mm. Well plate 1100 can have multiple holes along a perimeter of a top surface of the well plate 1100, for example one hole at each of the four corners, to receive a connector for attaching to a face plate 1300, as shown in FIG. 13. The holes can be configured so no thread is required. The holes can have a diameter of about 4 mm and be about, from a center of the hole, 3.5 mm from a length edge and a width edge of well plate 1100. The example well plate 1100 shown in FIG. 11 includes two apertures 1102 each centered along a length side of well plate 1100 and opposite each other. In some embodiments, well plate 1100 may include three or more apertures 1102. Apertures 1102 can be about 20 mm, as measured from a center of the apertures 1102, from either end length of well plate 1100, about 4.69 mm from a bottom edge of the length side, and have a diameter of about 3 mm. Well plate 1100 includes a well 1104 that recesses from a top surface of the well plate with a depth of about 3.13 mm and a diameter of about 23.1 mm.

Apertures 1102 can connect to well 1104 and form a pathway 622 for fluid, such as cell media 606, to flow between the apertures 1102 and in and out of well plate 1100, which can mimic blood flow through a blood vessel. Pathway 622 can have a height of about 3 mm at apertures 1102, extending inward from the apertures 1102 substantially parallel or parallel to the top and bottom surfaces of well plate 1100, where a bottom of pathway 622 is about 9.25 mm from the top surface of the well plate 1100 and about 6.13 mm from the bottom of well 1104 and a top of the pathway 622 is about 6.19 mm from the top surface of the well plate 1100. Pathway 622 can rise substantially perpendicularly or perpendicularly to the top and bottom surfaces of well plate 1100 to connect to well 1104, at which point the pathway can have a cross section expanding about 3.55 mm and be about 8.45 mm from a side surface of the well plate 1100.

FIG. 12 shows top, bottom, sides, cross-sectional and perspective views of an exemplary a cell culture region 1106 of the at least one well plate 1200 with a plurality of apertures 1102 configured to provide a laminar flow of media. Well plate 1200 can have a length and width of about 40 mm and a thickness or height of about 12.38 mm. Well plate 1200 can have a plurality of holes, such as a hole at each of the four corners of the well plate 1200, configured to receive connectors to connect to face plate 1300 (shown in FIG. 13). The holes can have a diameter of about 2.08 mm and be about 3.5 mm from a length side and width side of well plate 1200. The holes can be threaded (e.g., a UNC 4-40 tap with pitch diameter limit of H2). Pathway 622, well 1104 can be similar to those of well plate 1100. Unlike well plate 1100 in FIG. 11, well plate 1200 can have a recess 1202 configured to receive O-ring 202. Recess 1202 can have an outer diameter of about 32.2 mm, an inner diameter of about 25 mm, and be about 3.9 mm from a side of well plate 1200. Recess 1202 can have a depth of about 3 mm and a breadth of about 3.6 mm. Apertures 1102 can be threaded (e.g., a UNC 10-32 tap with pitch diameter limit of H3).

FIG. 13 shows top, side and perspective views of an exemplary face plate 1300. Face plate 1300 can have a length and width of about 40 mm and a thickness or height of about 3.13 mm. Face plate 1300 can be configured to removably attach to any well plate described herein, such as well plates 620, 1100, and 1200, via holes in the face plate 1300 that can receive connectors. The holes can be at each corner of a top surface of face plate 1300 and have a diameter of about 3.1 mm and be about 3.5 mm from a length side and a width side of the face plate 1300.

In an aspect of the presently disclosed subject matter, there is provided a method for providing continuous flow delivery to a cell culture. In some embodiments, the method comprises: providing a cell culture system, the system comprising at least one well plate configured to receive a cell culture wherein the at least one well plate comprises a plurality of apertures configured to provide a laminar flow of media; a face plate, wherein the face plate is configured to cover the at least one well plate and a cell culture region between the at least one well plate and the face plate, wherein the at least one well plate and the face plate are sealably connected to form a sealed chamber for the cell culture region where the cell culture is contained and is proximal to the laminar flow of media, wherein the system provides a continuous laminar flow of media to the cell culture; and flowing the laminar flow media to and/or from the cell culture region.

In some embodiments of the methods, the laminar flow of media comprises a media. In some embodiments of the methods, the media comprises any media one skilled in the art may use to support cell culture, storage and/or proliferation. In some embodiments of the methods, the media comprises universal culture media. In some embodiments of the methods, the media comprises blood mimic media. In some embodiments of the methods, the media comprises media with or without supplements. In some embodiments of the methods, the media is selected from the group consisting of glutamine, high glucose concentrations, low glucose concentrations, HEPES antibiotics, PenStrep, antimycotics, sodium pyruvate, sodium bicarbonate, Dulbecco's Modified Eagle Medium, RPMI 1640 Media, DMEM/F12 Media Formulations, Minimum Essential Medium (MEM) Ham's F-10 Media Formulation, Ham's F-12 Media Formulation, Medium 199 (M199), Eagle's Minimum Essential Medium, Iscove's Modified Dulbecco's Medium, Hybri-Care Medium, McCoy's 5A, Leibovitz's L-15 Medium and any combination thereof.

In some embodiments, the methods disclosed herein can further comprise additionally flowing a first media to a first cell culture region and the first media to a second cell culture region. In some embodiments, the method further comprises additionally flowing a first media to a first cell culture region and a second media to a second cell culture region. In some embodiments of the methods, the first cell culture region is separated from the second cell culture region by a cell-to-cell porous membrane. In some embodiments of the methods, the cell-to-cell porous membrane comprises a material selected from the group consisting of collagen, fibrin, laminin, fibronectin, vitronectin, silk, silk fibroin, decellularized extracellular matrices, decellularized extracellular matrices from specific tissues, Xylyx® bio tissue-specific extracellular matrix, protein extract from Engelbreth-Holm-Swarm (EHS) culture, ATCC® basement membrane extract, Cultrex® basement membrane extract, Matrigel® basement membrane extract, MaxGel™ basement membrane extract, GrowDex® basement membrane extract, ObaGel™ basement membrane extract, alginate, agarose, chitosan, gelatin, hyaluronic acid, nanocellulose, CorGel™ gel, GrowDex® saccharide or glycocalyx-derived gel, peptide-based hydrogels, polyethylene glycol (cross-linked) gels, polyacrylamide, polyacrylate and poly-methacrylate, polyvinyl alcohol, poly(lactic-co-glycolic acid), polylactic acid, gelatin methacrylate (GelMa), poly(ethylene glycol) diacrylate (PegDa) and any combination thereof.

In some embodiments of the methods, the at least one well plate and the face plate are made by a manufacture process. In some embodiments of the methods, the manufacture process is selected from the group consisting of additive manufacturing technologies, 3D printing, deposition molding, extrusion printing, photopolymerization, selective laser sintering, laser cutting, stereolithographic (SLA) printing, compression molding, injection molding, laser cutting, physical cutting, slicing, dye punching, hole punching, scissors, exact-o blading, rubber molding, thermal press with dyes, thermal press with stamps, transfer molding and any combination thereof. Representative techniques are also described in the Examples presented herein below.

In some embodiments of the methods, the at least one well plate and the face plate comprise a cell-compatible material. In some embodiments of the methods, the cell-compatible material is selected from the group consisting of nitrile (BUNA-N) rubber, ethylene propylene diene monomer (EPDM), fluorocarbon-based rubber, Viton, food-grade silicone, nitrile, polydimethylsiloxane (PDMS), polyethylene, high-density polyethylene (HDPE), low-density polyethylene (LDPE), polyetherketone (PEEK), polyethylene terephthalate (PET), polyethylene terephthalate glycol (PETG), polylactic acid, polypropylene, polystyrene, polytetrafluoroethylene (PTFE), Santoprene rubber, silicone and any combination thereof.

In some embodiments of the methods, the face plate is affixed to the at least one well plate. In some embodiments of the methods, the face plate is affixed to the at least one well plate by at least one screw. In some embodiments of the methods, the face plate is affixed to the at least one well plate by at least one clasp.

In some embodiments of the methods, the system further comprises an O-ring, wherein the O-ring provides a seal between the face plate and the at least one well plate defining the cell culture region. In some embodiments of the methods, the O-ring comprises a material selected from the group consisting of BUNA-N rubber, fluorocarbon-based rubbers, Viton, food-grade silicon, PDMS, polypropylene, Santoprene rubber, silicone. In some embodiments of the methods, the O-ring comprises silicone.

In some embodiments of the methods, the plurality of apertures comprises a plurality of tube fittings directing the laminar flow of media over the cell culture. In some embodiments of the methods, the plurality of tube fittings are configured to receive a tube providing the laminar flow of media.

In some embodiments of the methods, the cell culture is positioned between the at least one well plate and/or face plate. In some embodiments of the methods, the cell culture is positioned on the at least one well plate. In some embodiments of the methods, the cell culture is positioned on the face plate. In some embodiments of the methods, the cell culture transverses the at least one well plate and the face plate.

In some embodiments of the methods, the cell culture is embedded in a cell-compatible matrix, e.g., a hydrogel, optionally wherein the cell culture comprises a cell-laden hydrogel. In some embodiments of the methods, the cell-compatible matrix comprises a material selected from the group comprising protein- or peptide-based gels, saccharide-derived gels, glycocalyx-derived gels, synthetic hydrogels and any combination thereof. In some embodiments of the methods, the cell-compatible matrix comprises a material is selected from the group consisting of collagen, fibrin, laminin, fibronectin, vitronectin, silk, silk fibroin, decellularized extracellular matrices, extracellular matrices from decellularized tissues, Xylyx® bio tissue-specific extracellular matrix, protein extract from Engelbreth-Holm-Swarm (EHS) culture, ATCC® basement membrane extract, Cultrex® basement membrane extract, Matrigel® basement membrane extract, MaxGel™ basement membrane extract, GrowDex® basement membrane extract, ObaGel™ basement membrane extract, alginate, agarose, chitosan, gelatin, hyaluronic acid, nanocellulose, CorGel™ gel, GrowDex® saccharide or glycocalyx-derived gel, peptide-based hydrogels, polyethylene glycol (cross-linked) gels, polyacrylamide, polyacrylate and poly-methacrylate, polyvinyl alcohol, poly(lactic-co-glycolic acid), polylactic acid, gelatin methacrylate (GelMa), poly(ethylene glycol) diacrylate (PegDa) and any combination thereof.

In some embodiments of the methods, the cell culture region confines the cell culture to a specific location on the at least one well plate and/or face plate. In some embodiments of the methods, the cell culture region confines the cell compatible matrix to a specific location on the at least one well plate and/or face plate.

In some embodiments of the methods, the at least one well plate and the cell culture are attached to a second at least one well plate and a second cell culture with a cell-to-cell porous membrane separating the cell culture from the second cell culture. In some embodiments of the methods, a first laminar flow of media is connected to the at least one well plate and the first laminar flow of media is connected to the second at least one well plate. In some embodiments of the methods, a first laminar flow of media is connected to the at least one well plate and a second laminar flow of media is connected to the second at least one well plate.

In some embodiments of the methods, the cell culture region comprises an exchange media, where the flow is laminar after about 1 mL to about 50 mL of a media flows through the system. In some embodiments of the methods, the cell culture region comprises an exchange of media, where the flow is laminar after about 20 ml of a media flows through the system. In some embodiments of the methods, an optimum flow rate of about 1 mL/min to about 50 mL/min for the laminar flow of media. In some embodiments of the methods, an optimum flow rate of about 10.185 mL/min for the laminar flow of media.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Example 1: Manufacture

The continuous fluid delivery system (CFDS) was manufactured utilizing rapid and cost-effective small-scale manufacturing techniques. The base macrofluidic channel was fabricated utilizing Biomed clear resin on a Form 3B+ SLA 3D printer. The face and holder base plates were laser cut from a 3.125 mm sheet of acrylic, utilizing a Omtech 55W CO₂ laser cutter. The culture holders were 3D printed on a Qidi X-plus FDM 3D printer with ColorFab_XT filament. All of the materials utilized in this work have previously been shown to be biocompatible or have been USP class IV or VI certified, indicating biocompatibility. To help prevent bubbles formation in culture region, we utilized relatively large cross-sectional diameters and direct the medium up through the bottom of the flow cell against gravity, allowing bubbles to naturally clear from the device.

Within the CFDS the medium in the cell culture region is completely exchanged after approximately 20 ml of medium. The complete exchange of medium indicates that the entire culture area can be used for cell culture and will not result any diffusion-limited regions at the culture medium interface or differences between cultures within a single set-up. The exchange is pictorially represented below, with the initial solution being red food coloring and exchanging with water.

### Example 2: Testing

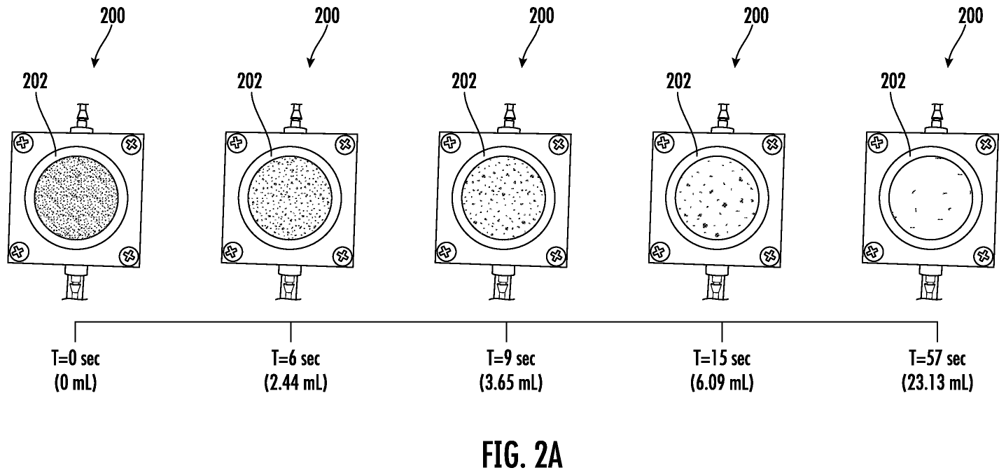

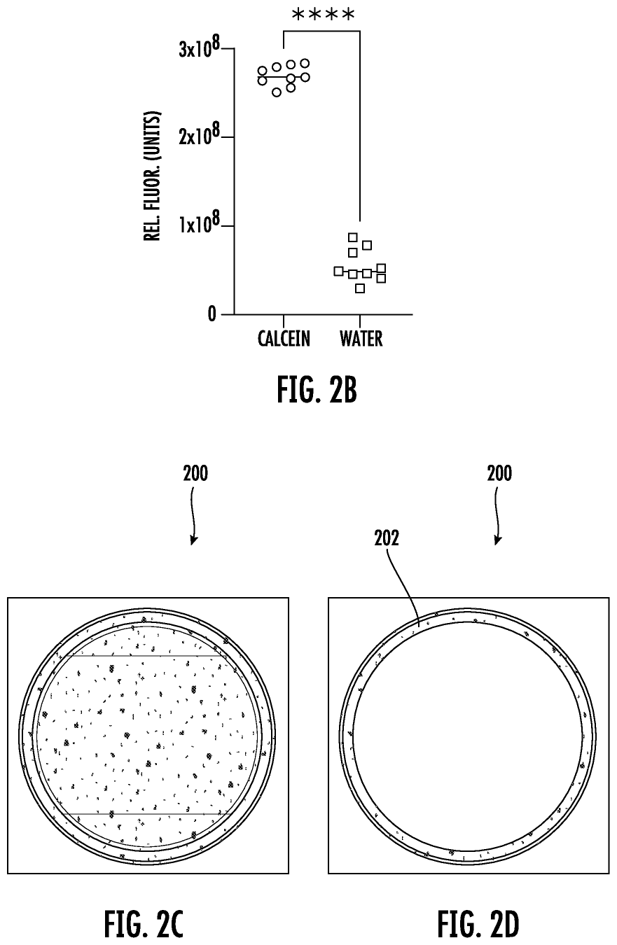

FIG. 2A shows a colorimetric evaluation of complete media exchange in a CFDS 200 after approximately 20 mL of medium while not creating any diffusion-limited regions indicating that the entire culture area can be utilized. FIG. 2B shows a quantitative evaluation of the complete removal of the medium by significant removal of fluorescent solution after 20 mL of flushing water. FIG. 2C shows fluorescent solution with 5 μM calcein in CFDS 200. A fluorescent solution was used to demonstrate the complete removal of the medium, pictured below. We alternated between an aqueous solution containing calcein dye (a fluorescent solution) and water to measure the intensity of the culture region after 20 ml of solution after switching. We found that the fluorescence was statistically reduced, indicated both pictorially and quantitatively. FIG. 2D shows remnants of the fluorescent solution in an O-ring 202 of CFDS 200 after H₂O flushes the 5 μM calcein. While not wishing to be bound by theory, it is believed that the reaming fluorescent artifact remaining after flushing with water is from non-specific binding of the calcein to O-ring 202. We believe that the only fluorescence remaining in the water sample is from the non-specific binding of the calcein to O-ring 202.

### Example 3: Flow Profile

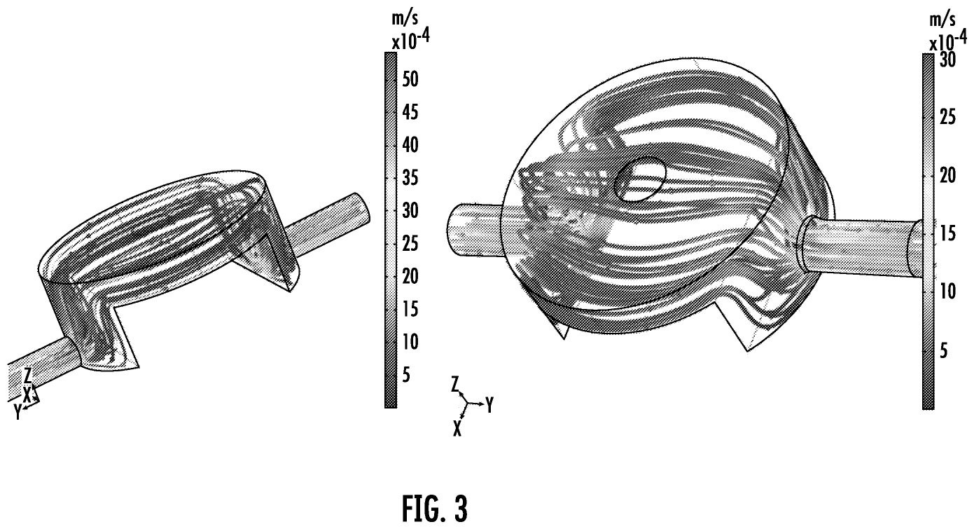

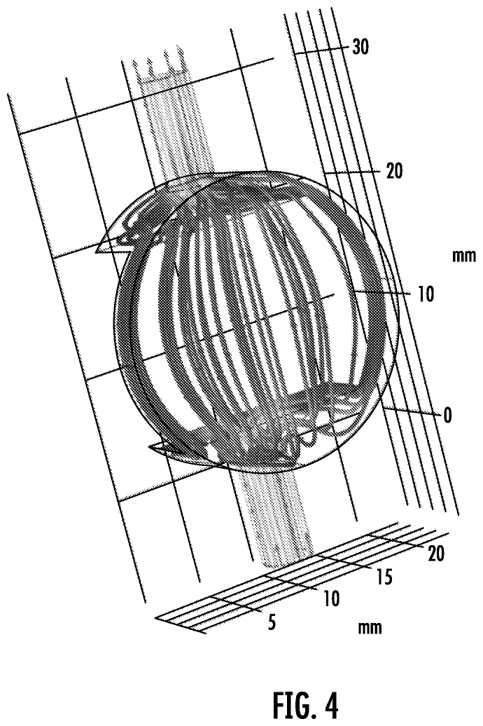

FIG. 3 shows a finite element simulation rendering to determine the flow profile in the culture device with streamlines and arrow lines representing velocity fields. To determine the flow profile in the culture device, the device was modeled using the computational fluid dynamics (CFD) finite element modeling package in COMSOL. We found that the flow profile demonstrates a laminar profile throughout the culture region. This finding was confirmed by a Reynolds number of approximately 20, indicating a laminar profile as well. FIG. 4 shows finite element simulation used to calculate the Reynolds Number in the culture device using the formula

$$Reynolds Number = \frac{ρ VD}{μ} ,$$

wherein μ=Fluid dynamic Viscosity, ρ=Fluid Density, V=Fluid Velocity, and D=Pipe Diameter. A Reynolds number greater than 3500 indicates turbulent flow, whereas a Reynolds number less than 3500 indicates laminar flow. A calculated Reynolds number was 19.91.

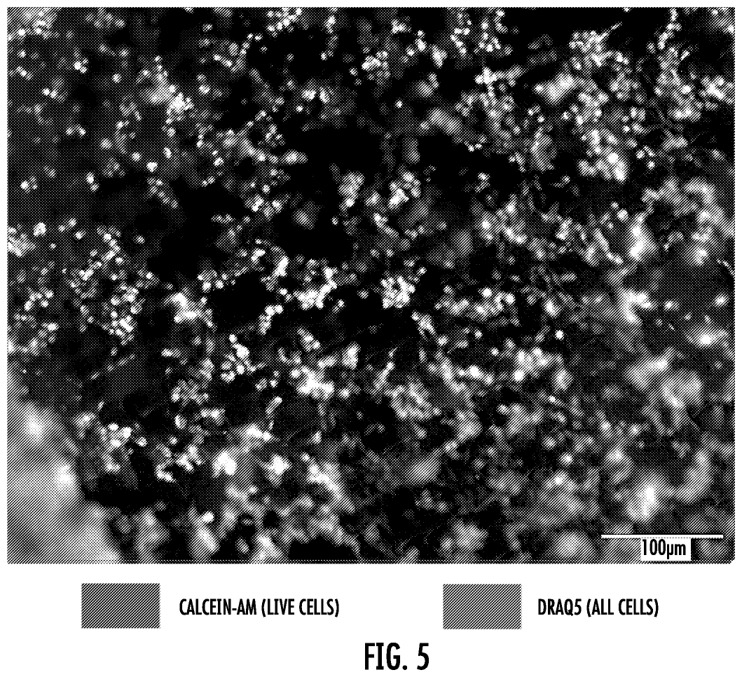

We found that a flow rate of 10.2 mL/min is optimal for this culture system, with the cells surviving for at least 3 days, with a measured viability of 93.8%. FIG. 5 provides a micrograph of the MDA-MB-231 breast adenocarcinoma cell line after 72 hours in CFDS 200, stained with calcein-AM (live cells) and DRAQ-5 (all cells). Viability was 93.8% and a rate flow was 10.19 mL/min. The flow rate of 10.2 mL/min indicates that the medium in the culture region is completely replaced approximately every 2 mins.

### Example 4: Stromal Factor Evaluation

When evaluating the role of stromal factors in drug resistance, the ability to evaluate the cellular responses in the presence and absence of prototypical gradients (e.g., pH, nutrients, O₂) is important to understanding the role oxygen plays. Previously, workflows with two scaffolds in the absence of gradients have been difficult to perform. We have found that from the possible experimental set-ups, the cells in one of the scaffolds die over the 72 hour incubation. However, with the CFDS we can culture two scaffolds in the absence of gradients without the cells dying. Below we show the experimental set-up for the culture and a comparison of the two scaffolds after 72 hours.

FIG. 6A shows a cross sectional schematic of CFDS 600 with two culture scaffolds 602. FIG. 6B is a chart of relative luminous units for side 1 and side 2 of the culture scaffolds 602, which demonstrates that the two culture scaffolds 602 can be performed in the absence of gradients without the cells dying with a comparison of the two cultures after 72 hours. CFDS 600 includes 3D printed BioMed clear material 616, which can be used to form one or more well plates 620 and/or a face plate 720 (shown in FIG. 7A). CFDS 600 can include one or more pathways 622 for cell media 606 to flow through and contact a porous filter, such as a dialysis sheet 612 or a filter paper 614 that mimic a basement membrane. CFDS 600 and/or CFDS 700 can also include tube fittings 604, cell-containing scaffolds 608 and 610, O-rings 202, and wax borders 618.

### Example 5: Basement Membrane Mimetics

By introducing different basement membrane mimics, the passing of signaling factors that promote invasion can be tuned based on the experimental needs. The CFDS can utilize materials such as 1) nylon filters with pore sizes ranging from 0.1 μm to 5 μm or 2) dialysis membranes with a molecular level cut off ranging from 1-100 KDa. The use of the 0.1 μm nylon filters allows us to significantly reduce invasion between the scaffolds, while the 5 μm nylon filters allows invasion of the cells between the scaffolds. We found that the nylon filter and dialysis membrane had no statistical difference in viability after 24 h. The dialysis membrane can be used to help increase the concentration of signaling factors within the culture set-ups. The 3.5 kDa cut-off allow nutrients and small molecules to pass through, however does not let anything with a size larger than 3.5 kDa in or out of the culture set-up. This effectively traps the signaling factors within the culture set-up and concentrates them, rather than allowing them to flow into the surrounding medium diluting the factors.

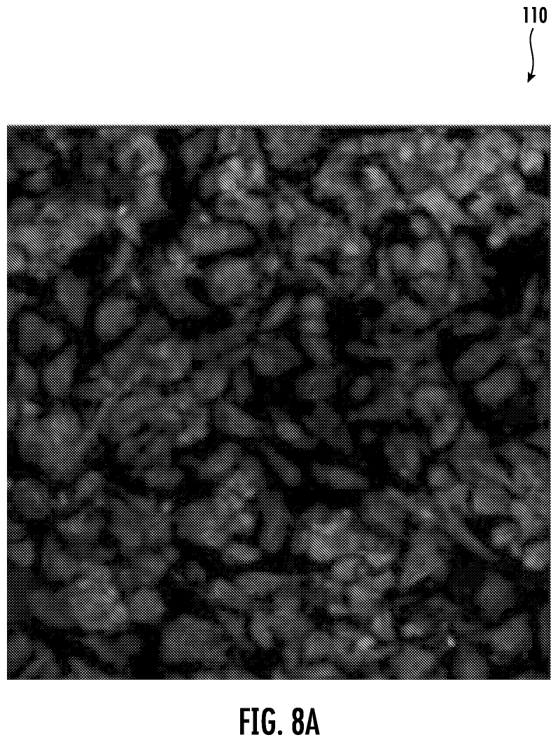

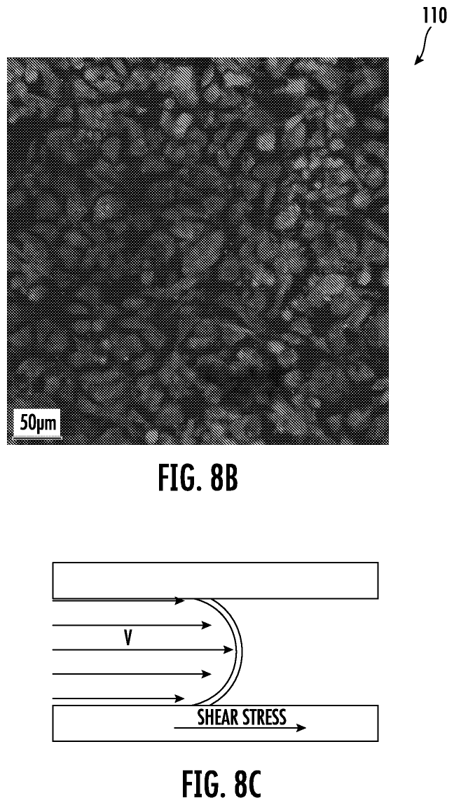

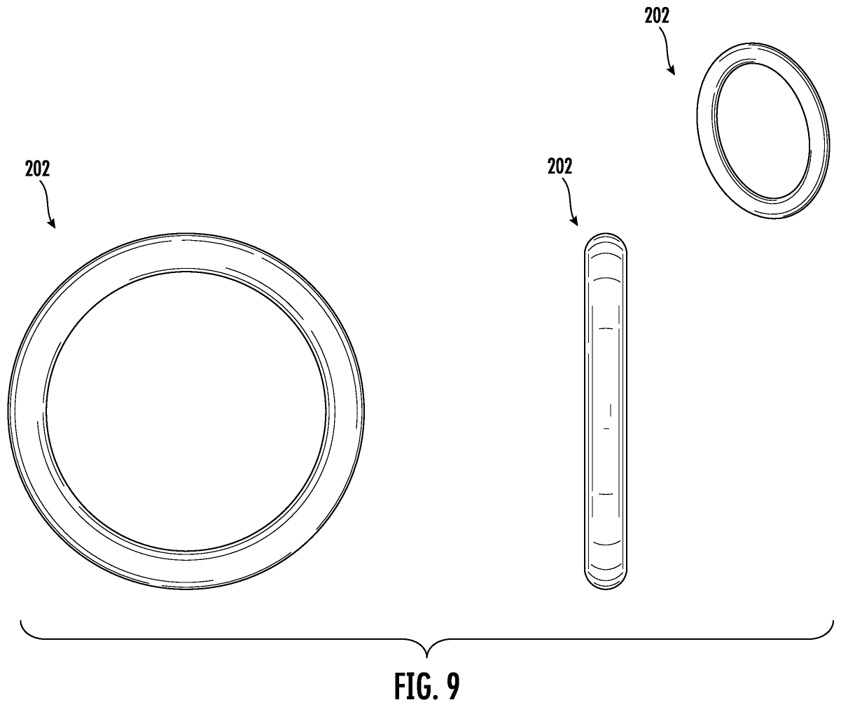

FIGS. 8A-8B are micrographs of endothelial cells cultured on a sheet of nylon filter paper, attached to the scaffold after 24 hours in the CFDS. FIG. 8C shows a schematic demonstrating endothelial cells 110 on the nylon filter paper exposed to flowing medium where the endothelial cells 110 experience a shear stress of 1.97 dyne/cm² where physiological shear stress in veins is 1-6 dyne/cm². The CFDS has allowed us to incorporate some of the structures that allow us to mimic blood vessels in our culture system. Primarily the CFDS has allowed us to incorporate endothelial cells (the cells that line the blood vessels) into our culture system.

In addition, these endothelial cells are cultured on a sheet of nylon filters with pore sizes of 0.1 μm, a size that matches the pore sizes of the basement membrane. This increases the physiological relevance of the models we are building, but also allows us to incorporate physiological stress onto the endothelial cells. In the CFDS the cells are exposed to flowing medium and thus a shear stress in a physiologically relevant manner. Physiologically the endothelial cells experience a shear stress of 1.88 Dyne/cm², while in the CFDS the endothelial cells experience a shear stress of approximately 1.98 Dyne/cm².

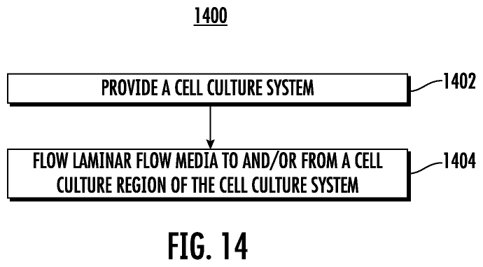

FIG. 14 is a flow diagram illustrating an example method of continuous flow delivery to a cell culture. At step 1402, a cell culture system is provided. The cell culture system can include at least one well plate configured to receive a cell culture wherein the at least one well plate comprises a plurality of apertures configured to provide a laminar flow of media. The cell culture system can further include a face plate, wherein the face plate is configured to cover the at least one well plate. The cell culture system can further include a cell culture region between the at least one well plate and the face plate, wherein the at least one well plate and the face plate are sealably connected to form a sealed chamber for the cell culture region where the cell culture is contained and is proximal to the laminar flow of media. The cell culture system can provide a continuous laminar flow of media to the cell culture.

The face plate can be affixed to the at least one well plate. The cell culture system can include an O-ring that provides a seal between the face plate and the at least one well plate defining the cell culture region. The plurality of apertures comprises a plurality of tube fittings directing the laminar flow of media over the cell culture. The cell culture can be embedded in a cell-compatible matrix, e.g., a hydrogel, optionally wherein the cell culture comprises a cell-laden hydrogel. The cell culture region can confine the cell culture to a specific location on the at least one well plate and/or face plate. The cell culture system can include a porous filter separating the cell culture from the laminar flow of media.

At step 1404, a laminar flow media is flowed to and/or from a cell culture region of the cell culture system. A first media can flow to a first cell culture region and a second cell culture region. A first media can flow to a first cell culture region and a second media can flow to a second cell culture region. The first cell culture region can be separated from the second cell culture region by a cell-to-cell porous membrane. The cell culture region can include an exchange of the laminar flow of the media after about 1 mL to about 50 mL of a media flows through the system. The cell culture region can include an exchange of the laminar flow of the media after about 20 ml of a media flows through the system. The laminar flow of media can include an optimum flow rate of about 1 mL/min to about 50 mL/min. The laminar flow of media can include an optimum flow rate of about 10.185 mL/min.

It will be understood that various details of the presently disclosed subject matter may be changed without departing from the scope of the presently disclosed subject matter. Furthermore, the foregoing description is for the purpose of illustration only, and not for the purpose of limitation.

1. A cell culture system, the system comprising:
at least one well plate configured to receive a cell culture wherein the at least one well plate comprises a plurality of apertures configured to provide a laminar flow of media;
a face plate, wherein the face plate is configured to cover the at least one well plate; and
a cell culture region between the at least one well plate and the face plate, wherein the at least one well plate and the face plate are sealably connected to form a sealed chamber for the cell culture region where the cell culture is contained and is proximal to the laminar flow of media,
wherein the system provides a continuous laminar flow of media to the cell culture.

2. The system of claim 1, wherein the face plate is affixed to the at least one well plate.

3. The system of claim 1, further comprising an O-ring, wherein the O-ring provides a seal between the face plate and the at least one well plate defining the cell culture region.

4. The system of claim 1, wherein the plurality of apertures comprises a plurality of tube fittings directing the laminar flow of media over the cell culture.

5. The system of claim 1, wherein the cell culture is positioned between the at least one well plate and/or face plate.

6. The system of claim 1, wherein the cell culture is embedded in a cell-compatible matrix.

7. The system of claim 1, wherein the cell culture region confines the cell culture to a specific location on the at least one well plate and/or face plate.

8. The system of claim 1, further comprising a porous filter separating the cell culture from the laminar flow of media.

9. The system of claim 1, wherein the at least one well plate and the cell culture are attached to a second at least one well plate and a second cell culture with a cell-to-cell porous membrane separating the cell culture from the second cell culture.

10. The system of claim 9, comprising a first laminar flow of media connected to the at least one well plate and the first laminar flow of media connected to the second at least one well plate.

11. The system of claim 9, comprising a first laminar flow of media connected to the at least one well plate and a second laminar flow of media connected to the second at least one well plate.

12. The system of claim 1, wherein the cell culture region comprises an exchange of the laminar flow of media after about 1 mL to about 50 mL of a media flows through the system.

13. The system of claim 1, wherein the cell culture region comprises an exchange of the laminar flow of media after about 20 mL of a media flows through the system.

14. The system of claim 1, comprises an optimum flow rate of about 1 mL/min to about 50 mL/min for the laminar flow of media.

15. The system of claim 1, comprises an optimum flow rate of about 10.185 mL/min for the laminar flow of media.

16. A method of continuous flow delivery to a cell culture, the method comprising:
providing the cell culture system of claim 1; and
flowing the laminar flow media to and/or from the cell culture region.

17. The method of claim 16, further comprising additionally flowing a first media to a first cell culture region and the first media to a second cell culture region.

18. The method of claim 16, further comprising additionally flowing a first media to a first cell culture region and a second media to a second cell culture region.

19. The method of claim 17, wherein the first cell culture region is separated from the second cell culture region by a cell-to-cell porous membrane.

20. The method of claim 16, wherein the face plate is affixed to the at least one well plate.

21. The method of claim 16, further comprising providing an O-ring, wherein the O-ring provides a seal between the face plate and the at least one well plate defining the cell culture region.

22. The method of claim 16, wherein the plurality of apertures comprises a plurality of tube fittings directing the laminar flow of media over the cell culture.

23. The method of claim 16, wherein the cell culture is embedded in a cell-compatible matrix.

24. The method of claim 16, wherein the cell culture region confines the cell culture to a specific location on the at least one well plate and/or face plate.

25. The method of claim 16, wherein the cell culture region confines the cell culture to a specific location on the at least one well plate and/or face plate.

26. The method of claim 16, further comprising providing a porous filter separating the cell culture from the laminar flow of media.

27. The method of claim 16, wherein the cell culture region comprises an exchange of the laminar flow of the media after about 1 mL to about 50 mL of a media flows through the system.

28. The method of claim 16, wherein the cell culture region comprises an exchange of the laminar flow of the media after about 20 mL of a media flows through the system.

29. The method of claim 16, wherein the laminar flow of media comprises an optimum flow rate of about 1 mL/min to about 50 mL/min.

30. The method of claim 16, wherein the laminar flow of media comprises an optimum flow rate of about 10.185 mL/min.

31. The method of claim 16, wherein the face place is optically transparent.

32. The system of claim 6, wherein the cell-compatible matrix is a hydrogel.

33. The system of claim 32, wherein the cell culture comprises a cell-laden hydrogel.

34. The method of claim 23, wherein the cell-compatible matrix is a hydrogel.

35. The method of claim 34, wherein the cell culture comprises a cell-laden hydrogel.
